(19) United States
(12) Patent Application Publication
(10) Pub. No.: US 2007/0185122 A1
(43) Pub. Date: Aug. 9, 2007

(54) Use of efletirizine for treating persistent allergic rhinitis

(76) Inventor: Christophe Revirron, Villarvolard (CH)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/557,448

(22) PCT Filed: May 24, 2004
(86) PCT No.: PCT/EP04/05572

§ 371 Date: Dec. 22, 2006

(30) Foreign Application Priority Data
May 28, 2003 (EP) 03011984.6

(51) Int. Cl. A61K 31/495

(52) U.S. Cl. 514/252.1

(57) ABSTRACT

The present invention relates to a pharmaceutical use of efletirizine for the treatment of persistent allergic rhinitis.

The present invention relates to the use of efletirizine for the preparation of drugs effective for the treatment of the persistent allergic rhinitis.

Efletirizine is known for the treatment of seasonal and perennial allergic rhinitis.

It has now surprisingly been found that efletirizine possesses therapeutic properties which render it particularly useful in the treatment of persistent allergic rhinitis.

The purpose of the invention concerns the treatment of persistent allergic rhinitis.

The present invention is based on the unexpected recognition that administration of pharmaceutical compositions comprising efletirizine, a crystalline form thereof or a pharmaceutically acceptable salt thereof to a patient treats the persistent allergic rhinitis.

The present invention encompasses a method for treating persistent allergic rhinitis which comprises administering to a patient a therapeutically effective amount of efletirizine, a crystalline form thereof or a pharmaceutically acceptable salt thereof.

The present invention also encompasses the use of efletirizine, a crystalline form thereof or a pharmaceutically acceptable salt thereof for the preparation of a medicament intended for the treatment of persistent allergic rhinitis.

The present invention relates to the use of efletirizine, a crystalline form thereof or a pharmaceutically acceptable salt thereof for the preparation of a medicament intended for decreasing the symptoms of persistent allergic rhinitis and improving the quality of life.

The term “efletirizine” as used herein refers to 2-[2-[4-[bis(4-fluorophenyl)methyl]-1-piperazinyl]ethoxy]acetic acid.

2-[2-[4-[Bis(4-fluorophenyl)methyl]-1-piperazinyl]ethoxy]acetic acid, also known and hereinafter referred to as efletirizine (INN: International Non-proprietary Name), is the compound of the following formula:

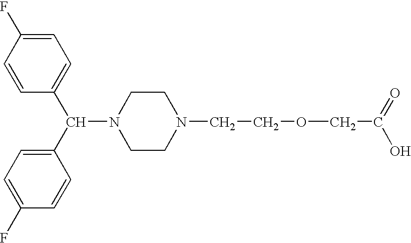

Efletirizine is encompassed within general formula I of European patent No. 58146 in the name of the applicant, which relates to substituted benzhydrylpiperazine derivatives.

Efletirizine has been found to possess excellent antihistamic properties. It belongs to the pharmacological class of histamine H₁-receptor antagonists and shows in vitro high affinity and selectivity for H₁-receptors. It is useful as an antiallergic, and antihistaminic agent.

The term “crystalline form” as used herein refers to any pseudopolymorphic or polymorphic form of efletirizine and, in particular, to the two pseudopolymorphic crystalline forms of efletirizine dihydrochloride, namely anhydrous efletirizine dihydrochloride and efletirizine dihydrochloride monohydrate which are described in the European patent No. 1 034 171, and also to another pseudopolymorphic form of efletirizine dihydrochloride which is described in the international patent application WO 03/009849.

Processes for preparing efletirizine or a pharmaceutically acceptable salt thereof have been described in European Patent 1 034 171, and in the international patent applications WO 97/37982 and WO 03/009849.

The term “pharmaceutically acceptable salts” as used herein refers not only to addition salts with pharmaceutically acceptable non-toxic organic and inorganic acids, such as acetic, citric, maleic, succinic, ascorbic, hydrochloric, hydrobromic, sulfuric, and phosphoric acids and the like, but also its metal salts (for example sodium or potassium salts) or ammonium salts, the amine salts and the aminoacid salts. The best results have been obtained with efletirizine dihydrochloride.

By patient, we understand infants, children, adolescents and adults.

By the term “allergic rhinitis”, we understand a symptomatic disorder of the nose induced by an IgE-mediated inflammation after allergen exposure of the membrane of the nose. Symptoms of allergic rhinitis include rhinorrhea, nasal obstruction, nasal itching, sneezing, ocular pruritis. The term “persistent allergic rhinitis”, as used herein, refers to a disease when symptoms last more than 4 days per week and for more than 4 weeks. It is subdivided into mild and moderate-severe rhinitis. It is said “mild” when there are normal sleep, or no impairment of normal daily activities, sport, leisure, normal work and school, or no troublesome symptoms. It is said “moderate-severe” when there are abnormal sleep, or impairment of daily activities, sport, leisure, or problems caused at work or school, or troublesome symptoms.

A therapeutically effective amount of efletirizine or a pharmaceutically acceptable salt thereof is used to treat or alleviate the effects of persistent allergic rhinitis. The dosage depends essentially on the specific method of administration and on the purpose of the treatment and on the severity of the disease. The size of the individual doses and the administration program can best be determined based on an individual assessment of the relevant case. The methods required to determine the relevant factors are familiar to the expert.

A preferred daily dosage provides from about 0.01 mg to about 5 mg of efletirizine or a pharmaceutically acceptable salt thereof, per kg of body weight per patient. A particularly preferred daily dosage is from about 0.1 to about 3 mg per kg of body weight per patient. The best results have been obtained with a daily dosage from about 0.1 to 2 mg per kg of body weight per patient. The dosage may be administered once per day of treatment, or divided into smaller dosages, for examples 1 to 4 times a day, and preferably 1 to 3 times a day, and administrated over about a 24 hours time period to reach a total given dosage. The exact dosages in which the compositions are administrated can vary according to the type of use, the mode of use, the requirements of the patient, as determined by a skilled practitioner. The exact dosage for a patient may be specifically adapted by a skilled person in view of the severity of the condition, the specific formulation used, and other drugs which may be involved.

Pharmaceutical compositions used according to the present invention may be administered by any conventional means. The routes of administration include intradermal, transdermal, intramuscular, oral, ocular, rectal and intranasal routes. Any other convenient route of administration can be used, for example absorption through epithelial or mucocutaneous linings.

Pharmaceutical compositions used according to the present invention may be immediate release dosage form, or slow release dosage form.

The pharmaceutical forms according to the present invention may be prepared according to conventional methods used by pharmacists. The forms can be administered together with other components or biologically active agents, pharmaceutically acceptable excipients, such as surfactants, carriers, diluents and vehicles.

The pharmaceutical compositions of the invention include any conventional therapeutical inert carrier. The pharmaceutical compositions can contain inert as well as pharmacodynamically active additives. Liquid compositions can for example take the form of a sterile solution which is miscible with water. Furthermore, substances conventionally used as preserving, stabilizing, moisture-retaining, and emulsifying agents as well as substances such as salts for varying the osmotic pressure, substances for varying pH such as buffers, and other additives can also be present. If desired an antioxidant can be included in the pharmaceutical compositions. Pharmaceutical acceptable excipients or carriers for compositions include saline, buffered saline, dextrose or water. Compositions may also comprise specific stabilizing agents such as sugars, including mannose and mannitol. Carrier substances and diluents can be organic or inorganic substances, for example water, gelatine, lactose, starch, magnesium stearate, talc, gum arabic, polyalkylene glycol and the like. A prerequisite is that all adjuvants and substances used in the manufacture of the pharmaceutical compositions are nontoxic.

Pharmaceutical compositions can be administered by spray inhalation. Any conventional pharmaceutical composition for spray inhalation administration may be used. Another preferred mode of administration is by aerosol.

The pharmaceutical composition of the invention can also be formulated for topical application. The composition for topical application can be in the form of an aqueous solution, lotion or jelly, an oily solution or suspension or a fatty or emulsion ointment.

The pharmaceutical composition of the invention can also be used for slow prolonged release with a transdermal or intramuscular therapeutic system or with an appropriate formulation for oral slow release.

The pharmaceutical compositions according to the present invention may also be administered orally or rectally. They may also be administered by nasal instillation, aerosols or in the form of unguents or creams. The pharmaceutical compositions which can be used for oral administration may be solid or liquid, for example, in the form of uncoated or coated tablets, pills, dragees, gelatine capsules, solutions, syrups and the like. For administration by the rectal route, the compositions containing the compounds of the present invention are generally used in the form of suppositories.

The pharmaceutical forms, such as tablets, capsules, pellets, drops, eye drops, suppositories and the like, are prepared by conventional pharmaceutical methods. The compounds of the present invention are mixed with a solid or liquid, non-toxic and pharmaceutically acceptable carrier and possibly also mixed with a dispersing agent, a disintegration agent, a stabilizing agent and the like. If appropriate, it is also possible to add preservations, sweeteners, coloring agents and the like.

Preferably, the pharmaceutical compositions of the invention is administered in traditional form for oral administration, as film coated tablets, capsules, dragees, and oral liquid preparation such as syrup.

As an Example of a composition according to the present invention, the following formulation of a film coated tablet is preferred: efletirizine dihydrochloride, magnesium stearate, cellulose, lactose, croscarmellose, and silicon dioxide.

As an Example of a composition according to the present invention, the following formulation of a syrup is preferred: efletirizine dihydrochloride, methyl- and propylparaben, saccharinum, and purified water.

Pharmaceutical compositions of the invention are useful to treat the persistent allergic rhinitis. These compositions can alleviate the effects of the persistent allergic rhinitis.

Another advantage of the invention is the ability of the process to improve quality of life and all symptoms of persistent allergic rhinitis.

The method of the invention is believed particularly suited to use in patients susceptible to suffer from persistent allergic rhinitis.

Another advantage of the invention is that efletirizine dihydrochloride has an effect on persistent rhinitis for more than 4 weeks.

It is shown that efletirizine dihydrochloride has an effect on quality of life for more than 4 weeks.

It is shown that efletirizine dihydrochloride has an effect on nasal congestion.

The invention is further defined by reference to the following example.

### EXAMPLE

A study relative to the clinical effect of efletirizine dihydrochloride is planed to establish on the intention to treat (ITT population) whether an approximately 6 month efletirizine treatment can improve the quality of life and clinical symptoms from adult patients suffering from persistent allergic rhinitis, when compared to placebo.

Secondary parameters of efficacy include different durations of treatment, different symptoms, different quality of life questionnaires, the incidence of co-morbidities suspected to be linked to allergic rhinitis and pharmaco-economic variables. The safety of this long-term treatment with efletirizine is also evaluated.

The target population of this study consists of adults aged more than 18 years suffering from persistent allergic rhinitis [WHO Initiative on Allergic Rhinitis and its Impact on Asthma (ARIA), 2000, pages S147-S149]. To be enrolled, the subjects need to have sufficient rhinitis symptoms during the selection period. Excluded are patients with ENT (Ear-Nose-Throat) or eye infection preceding initial visit.

The study is a prospective, randomized, double blind, parallel group, and placebo-controlled study with efletirizine dihydrochloride.

The severity of clinical symptoms is rated by the T5SS (sneezing, rhinorrhea, nasal pruritus, ocular pruritus and nasal congestion) evaluated each by a score from 0 to 3 each day. The impact on health related quality of life is measured using the Rhinoconjunctivitis Quality of Life Questionnaire (RQLQ, SF36) (E. JUNIPER and G. H. GUYATT, Development and testing of a new measure of health status for clinical trials in rhinoconjunctivitis, Clinical and Experimental Allergy 1991; 21:77-83; E. JUNIPER, Measuring Health Related Quality of Life in rhinitis, J. Allergy Clin. Immunol. 1997; 99:S742-9).

Study treatments last for approximately 6 months.

The primary end-point for efficacy is a decrease of the T5SS over the first 4 weeks.

Secondary parameters of efficacy include the mean T5SS, the RQLQ and the SF-36 questionnaire (Medical Outcomes Survey Short Form 36) at the different time points of the study, and the incidence and the duration of rescue medication over 6 months.

Exploratory parameters of efficacy include the mean of each individual rhinitis score, each RQLQ domain and each scale of the SF-36 questionnaire at the different time points of the study, the Global Evaluation Scale after 4 weeks and 6 months, the incidence of co-morbidities suspected to be linked to allergic rhinitis and the pharmaco-economic direct and indirect costs over 6 months.

At each of the visits, diary book entries (T5SS, RQLQ, SF-36, indirect cost pharmaco-economic parameters, concomitant medication, outpatient consultations and adverse events) are verified and transferred into the Clinical Record Form and direct cost pharmaco-economic parameters will be recorded. Patients have a physical examination, including the measurement of vital signs. At the beginning and at the end of the study they also do a safety lab test, including pregnancy test for females, and at Visits 4 and 7, they filled-in a Global evaluation scale.

Adverse events are recorded by the patients on diary cards and discussed with the investigator at each visit. Serious adverse events have to be reported immediately.

The aim is to demonstrate that efletirizine is able to treat persistent allergic rhinitis as long as it is administered, but also able to modify daily activities of patients, going beyond the simple symptom relief observed in short duration trials so far.

1-8. (canceled)

9. A method of treating a subject having persistent allergic rhinitis, the method comprising administering to the subject an effective amount of a composition comprising efletirizine, a crystalline form thereof, or a pharmaceutically acceptable salt thereof.

10. A method of decreasing the symptoms of persistent allergic rhinitis in a subject, the method comprising administering to the subject an effective amount of a composition comprising efletirizine, a crystalline form thereof or a pharmaceutically acceptable salt thereof.

11. A method of treating a subject having rhinorrhea, the method comprising administering to the subject an effective amount of a composition comprising efletirizine, a crystalline form thereof or a pharmaceutically acceptable salt thereof.

12. A method of treating a subject having nasal obstructions, the method comprising administering to the subject an effective amount of a compositing comprising efletirizine, a crystalline form thereof or a pharmaceutically acceptable salt thereof.

13. A method of treating nasal itching in a subject, the method comprising administering to the subject an effective amount of a composition comprising efletirizine, a crystalline form thereof or a pharmaceutically acceptable salt thereof.

14. A method of treating sneezing in a subject, the method comprising administering to the subject an effective amount of a composition comprising efletirizine, a crystalline form thereof or a pharmaceutically acceptable salt thereof.

15. A method of treating a subject having ocular pruritis, the method comprising administering to the subject an effective amount of a composition comprising efletirizine, a crystalline form thereof or a pharmaceutically acceptable salt thereof.

16. The method according to claim 9, wherein the salt is efletirizine dihydrochloride.

17. The method according to claim 10, wherein the salt is efletirizine dihydrochloride.

18. The method according to claim 11, wherein the salt is efletirizine dihydrochloride.

19. The method according to claim 12, wherein the salt is efletirizine dihydrochloride.

20. The method according to claim 13, wherein the salt is efletirizine dihydrochloride.

21. The method according to claim 14, wherein the salt is efletirizine dihydrochloride.

22. The method of use according to claim 15, wherein the salt is efletirizine dihydrochloride.
